(19) United States
(12) Patent Application Publication
(10) Pub. No.: US 2012/0046231 A1
(43) Pub. Date: Feb. 23, 2012

(54) COMPOSITION AND PROCESS FOR SYNTHESIZING POLYMERIZED HUMAN SERUM ALBUMIN FOR APPLICATIONS IN TRANSFUSION MEDICINE

(76) Inventors: Andre Francis Palmer, Bexley, OH (US); Pedro Cabrales, San Diego, CA (US)

(21) Appl. No.: 13/215,473

(22) Filed: Aug. 23, 2011

Related U.S. Application Data
(60) Provisional application No. 61/376,041, filed on Aug. 23, 2010.

(51) Int. Cl. A61K 38/38; A61P 7/00; C07K 14/765

(52) U.S. Cl. 514/15.2; 530/363; 530/364

(57) ABSTRACT

Described herein is a composition and process for synthesizing a human serum albumin (HSA) based plasma replacement composition that includes a polymerized HSA (PolyHSA) that is chemically stabilized by the reduction of Schiff bases. The PolyHSA may have a molecular weight of at least about 100 kDa and may optionally have a cross-linker to HSA molar ratio of at least about 10:1. The PolyHSA composition is useful for restoring a subject's circulatory volume.

### CROSS-REFERENCE TO RELATED APPLICATION

This application claims the filing benefit of U.S. Provisional Patent Application Ser. No. 61/376,041 filed Aug. 23, 2010, the disclosure of which is hereby expressly incorporated by reference herein in its entirety.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Grant No. R01HL078840 and R01DK070862 awarded by the National Institutes of Health.

### FIELD

The present invention relates generally to solutions to be used in transfusion medicine and more particularly to polymerized human serum albumin for use in transfusion medicine.

### BACKGROUND

When blood is unavailable, plasma replacement compositions are commonly used to treat patients with significant blood loss by restoring their circulatory volume. Plasma expanders (PEs) are a type of plasma replacement composition. PEs increase the oncotic pressure (also known as the colloid osmotic pressure, COP) of the plasma, by drawing fluid from the tissue space into the circulatory system. The resulting increase in blood volume and capillary pressure stabilizes the patient by restoring microvascular perfusion through the capillaries. Rather than attempting to restore the oxygen carrying capacity of blood as in the case of blood transfusions, PEs enhance oxygen delivery of the remaining RBCs in the blood by maintaining blood volume and sustaining adequate blood flow. The subsequent decrease in red blood cell (RBC) concentration also increases the shear rate, initiating a signaling cascade that dilates the blood vessels and sustains systemic oxygen delivery.

Several different PEs are currently in clinical use, including crystalloids based on saline (0.9%) and colloids like gelatin, dextran, hydroxyethyl starch (HES), and monomeric human serum albumin (HSA). The use of each of these materials has advantages and disadvantages. Saline based solutions are inexpensive, but their effects are short-lived, and must be continually supplied or eventually supplemented with another colloidal PE. Cross-linked gelatin is a readily available colloid, but it has poor volume expansion and is frequently associated with allergic reactions and edema. Synthetic colloids, such as dextran polymers and HES, are able to effectively restore circulatory volume and microvascular perfusion. Unfortunately, dextran and HES have both been shown to inhibit coagulation, aggregate RBCs, and lead to renal failure.

Monomeric HSA is an attractive PE for several reasons. It is naturally produced in the liver and secreted into the bloodstream at a high concentration (HSA comprises˜50% of the total plasma protein), where it binds a variety of drugs, toxic species, metabolic byproducts and other compounds and provides˜75% of the plasma's COP. Monomeric HSA also has desirable antioxidant properties, inhibits inflammation during resuscitation, and has been shown to increase vascular integrity, thereby limiting extravasation of itself and other plasma proteins. Despite all of these beneficial properties, clinical trials and meta analyses have provided some contradicting results. In most cases the use of monomeric HSA as a PE has been shown to be generally safe, with little to no increased risk of death. However, monomeric HSA has been shown to increase the risk of death in patients with severe burns or sepsis. This is most likely due to the increased vascular permeability and subsequent extravasation of monomeric HSA into the intravascular space caused by severe trauma or sepsis. Extravasation of monomeric HSA can also cause edema, reduce plasma COP, and expose tissues to any toxins bound to HSA. Therefore there is a need to synthesize large polymerized HSA (PolyHSA) molecules that are unable to extravasate through blood vessels in patients with both normal and compromised endothelia. Moreover, PolyHSA molecules that are not chemically stabilized will hydrolyze back into the monomeric form and will increase the risk of death in patients with severe burns or sepsis. Thus, there is a need for the development of a chemically stabilized PolyHSA-based PE that does not increase patient mortality.

PEs lower the systemic hematocrit (Hct) via hemodilution and decrease the oxygen carrying capacity of blood, which changes its rheological properties. The compensatory mechanisms that respond to the acute decrease in Hct involve the increase of cardiac output (due to the reduction in vascular resistance), which also partially recovers tissue oxygenation. Blood viscosity is an important factor that regulates the responses of the cardiovascular system, as it affects shear stress and activates the synthesis of vascular relaxation mediators such as nitric oxide (NO). NO is a critical regulator of basal blood vessel tone and vascular homeostasis, anti-platelet activity, modulation of endothelial and smooth muscle proliferation, and adhesion molecule expression. From a rheological standpoint, an acute decrease in Hct paired with an increase in plasma viscosity with high viscosity PEs can partially preserve whole blood viscosity. Based on this model, increasing plasma viscosity with a high viscosity PE can restore endothelial shear stress to the magnitude attained with non-diluted blood, without the need to fully restore blood viscosity and vascular resistance. High viscosity PEs significantly improved microvascular function in animal models of extreme hemodilution and organ blood flow compared with low viscosity PEs. Since the vascular system is coupled to the heart, improvement of microvascular function may accompany enhancement of cardiac function. Thus, there is a need for a safe and effective high viscosity plasma replacement composition. Hence, PolyHSA solutions should fulfill this need for a high viscosity plasma replacement composition.

### SUMMARY

In order to restore circulatory volume and to limit the detrimental side effects of currently available PEs and monomeric HSA, the size (i.e., molecular weight [MW]) of HSA should be increased in order to lower the extravasation of HSA. Thus, described herein are high MW (at least about 100 kDa) PolyHSA compositions that are chemically stabilized by reduction of Schiff bases. The stabilized PolyHSA compositions may have a cross-linker to HSA molar ratio of at least 10:1. The viscosity of PolyHSA compositions is higher than that of unpolymerized HSA compositions, when formulated at the same total protein concentration. Methods of using the PolyHSA compositions to restore circulatory volume in a subject are also described.

### BRIEF DESCRIPTION OF THE DRAWINGS

FIG. 1 is an image of an SDS-PAGE of native HSA and PolyHSA solutions according to embodiments of the invention.

FIG. 2 is a graph demonstrating the MW distribution of native HSA and PolyHSA solutions according to embodiments of the invention.

FIG. 3 is a graph demonstrating the circular dichroism spectra for native HSA and PolyHSA solutions according to embodiments of the invention.

FIG. 4 includes a bar graph and photomicrographs demonstrating RBC aggregation caused by native HSA, 500 kDa dextran, and PolyHSA solutions according to embodiments of the invention.

### DETAILED DESCRIPTION

An embodiment of the invention is directed to a composition of PolyHSA that is stabilized by reduction of Schiff bases and is useful in a plasma replacement composition. The PolyHSA has a MW of at least about 100 kDa. In one embodiment, a majority of the PolyHSA in the composition has a high MW of at least about 200 kDa and in another embodiment the high MW is at least about 2,000 kDa or at least about 10,000 kDa. The high MW PolyHSA compositions may have an upper limit of about 50,000 kDa. In one embodiment, more than 50% of the PolyHSA has a high MW (i.e. MW>MW of HSA) and in other embodiments, at least about 75% of the PolyHSA has a high MW, or at least about 85% of the PolyHSA has a high MW, or at least about 95% of the PolyHSA has a high MW, or at least about 100% of the PolyHSA has a high MW.

In addition, the PolyHSA may have a cross-linker to HSA molar ratio, referred to herein as the cross-link density, of at least about 10:1, or at least about 50:1, or at least about 100:1, and may optionally range between any of these cross-linking densities. For example, the cross-linking density may be in a range from about 10:1 to about 100:1. The MW and/or cross-link density of the PolyHSA compositions affect their biophysical characteristics, which directly determine viscosity and colloid osmotic pressure. As shown in the examples below, high MW PolyHSA compositions having higher cross-link densities generally have improved biophysical characteristics relative to native HSA and dextran.

The PolyHSA compositions have a higher viscosity than monomeric HSA compositions, when formulated at the same protein concentration. In one embodiment, the viscosity of the PolyHSA composition is about 1.1 times greater than the viscosity of the monomeric HSA composition having the same concentration. In another embodiment, the viscosity of the PolyHSA composition is about 8 times greater than the viscosity of the monomeric HSA composition having the same concentration. In another embodiment, the viscosity of the PolyHSA composition is about 10 times greater than the viscosity of the monomeric HSA composition having the same concentration.

The PolyHSA compositions have a lower COP than monomeric compositions at the same concentration level. In one embodiment, the COP of the PolyHSA composition is about ½ the COP of monomeric HSA. In another embodiment, the COP of the PolyHSA composition is about 1/10 the COP of monomeric HSA. In another embodiment, the COP is about 1/50 the COP of monomeric HSA.

Another aspect of the invention is directed to a process for synthesizing the PolyHSA compositions described above, i.e. a PolyHSA that is stabilized by the reduction of Schiff bases and is useful in plasma replacement compositions. The PolyHSA may have a MW of at least about 100 kDa and a cross-link density of at least about 10:1. The process includes polymerizing monomeric HSA with a cross-linker, quenching the polymerization reaction with a reducing agent, and collecting the PolyHSA having the desired MW.

Monomeric HSA useful in the present invention may come from any source such as HSA isolated from human serum using known techniques or recombinant HSA. The monomeric HSA is diluted or concentrated to the desired level, such as to 25 mg/mL with a suitable buffer. The polymerization reaction is initiated by the addition of a cross-linker, such as a 70% glutaraldehyde solution, to the HSA solution at the desired molar ratio of cross-linker to HSA: such as at least about 10:1, at least about 50:1, and at least about 100:1. The cross-linking density of the resulting PolyHSA composition may be controlled by controlling this molar ratio or by controlling the parameters of the polymerization reaction, such as the duration and temperature of the reaction. The cross-link density of a PolyHSA composition can be confirmed by separating PolyHSA from any free cross-linker after the polymerization reaction and quantifying the amount of free cross-linker compared to the initial amount of cross-linker used in the reaction. The difference between the two quantities would be equivalent to the amount of cross-linker that is cross-linked to the protein. Glutaraldehyde, like many cross-linkers, reacts with lysine, histidine, tyrosine, arginine, and primary amine groups, forming both intra and intermolecular cross-links within HSA and between neighboring HSA molecules in solution. Therefore, cross-linked HSA compositions include polymers of various MWs.

Cross-linkers in addition to glutaraldehyde include succindialdehyde, activated forms of polyoxyethylene and dextran, α-hydroxy aldehydes, such as glycolaldehyde, N-maleimido-6-aminocaproyl-(2′-nitro,4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl)butyrate, sulfosuccinimidyl 4-(p-maleimidophenyl)butyrate, 1-ethyl-3-(3-dimethylarninopropyl)carbodiimide hydrochloride, N,N′-phenylene dimaleimide, and compounds belonging to the bisimidate class, the acyl diazide class or the aryl dihalide class, and combinations thereof.

The HSA is allowed to polymerize with the cross-linker for a suitable period of time to obtain HSA having the desired MW. For example, the polymerization reaction may be incubated at about 37° C. for between 1 and 4 hours. The polymerization reaction is then quenched with a molar excess of reducing agent, preferably a strong reducing agent that is capable of reducing the Schiff bases in the PolyHSA and any remaining free aldehyde groups on the cross-linker. For example, the reaction may be quenched by incubating the reaction mixture with a 1 M sodium borohydride solution for 30 min at 37° C. Quenching the Schiff bases in the PolyHSA stabilizes the polymer and prevents the hydrolysis of PolyHSA back to monomeric HSA, which could extravasate and cause detrimental side effects. Moreover, reducing the aldehyde group on the cross-linker completely quenches the polymerization reaction. An exemplary strong reducing agent capable for use in embodiments of the invention is sodium borohydride, however it is understood that other reducing agents may be useful as well.

The MW distribution of the PolyHSA in the quenched reaction mixture will be affected by the conditions under which the polymerization reaction is conducted, such as duration and temperature of the incubation along with the cross-linker to HSA molar ratio. To control for variables in the polymerization reaction that might result in PolyHSA having a MW outside of the desired range, the process further includes the step of collecting PolyHSA having the desired MW range. The PolyHSA has a MW of least about 100 kDa and an upper limit of about 50,000 kDa. The collecting step may include separating or purifying PolyHSA having the desired MW range or making the PolyHSA free from undesirable elements such as HSA having a MW outside of the desired range. For example, the PolyHSA solution may be clarified such as by being passed through a glass chromatography column packed with glass wool to remove large particles. The clarified PolyHSA solution is then separated into distinct molecular mass fractions using known separation methods such as passing the clarified PolyHSA solution through a tangential flow filtration (TFF) hollow fiber (HF) cartridge selected to collect PolyHSA having the desired MW. For example, fractionation of the PolyHSA composition with a 100 kDa TFF HF cartridge (Spectrum Labs, Rancho Dominguez, Calif.) will result in the retentate containing PolyHSA molecules that are at least 100 kDa or larger and that fall within the desired MW in one embodiment of the invention. In that example, the filtrate will mostly contain PolyHSA molecules that are smaller than 100 kDa, i.e., molecules that are smaller than the desired MW. The MW of the PolyHSA can be controlled by passing the clarified PolyHSA solution through TFF HF cartridges having different pore sizes selective for the desired MW.

The PolyHSA solution may then be subjected to as many cycles of diafiltration with an appropriate buffer as needed in order to remove impurities having a MW outside of the desired range. The PolyHSA solution may also buffer exchanged to remove impurities such as unpolymerized cross-linkers and quenching agents which may be cytotoxic. After separation of the desired fraction, the filtrate may subsequently be concentrated such as with a 100 kDa TFF HF cartridge (Spectrum Labs). The MW distribution of the PolyHSA may be confirmed by known methods such as SDS-PAGE analysis or size exclusion chromatography coupled with multi-angle static light scattering.

In use, PolyHSA is utilized as a plasma replacement composition such as a PE to restore the capacity of the circulatory system to perfuse tissues during a hypovolemic crisis without the substantial side effects that can result from other PE compositions. For this use, a PolyHSA composition is infused into the circulatory system of the subject in a volume sufficient to restore the capacity of the circulatory system to perfuse tissues during a hypovolemic crisis, such as through intravenous or intraarterial infusion through a catheter. The PolyHSA composition may also be mixed with blood compositions, with includes whole blood, plasma, or blood fractions, as well as, crystalloid solutions and other PEs prior to infusion into the subject. The volume of the PolyHSA composition infused will vary depending on the degree of hypovolemia in the subject. In any event, the volume of the PolyHSA composition infused may be similar to the volume of other PE compositions, blood, or other plasma replacement compositions infused under similar conditions. In this regard, exemplary conditions in which PolyHSA may be useful include: treatments for wounds, detoxification of blood, anemia, head injury, hemorrhage, hypovolemia, ischemia, sickle cell crisis and stroke; enhancing cancer treatments; enhancing cell/organ/tissue preservation; alleviating cardiogenic shock; shock resuscitation; and cosmetics.

### Example

The following is a description of the specific method used to produce and test compositions in which a majority of the high MW PolyHSA has a MW of at least about 100 kDa and that are stabilized by reduction of Schiff bases.

Glutaraldehyde Polymerization of HSA—Albuminar®-25 (HSA) was purchased from ABO Pharmaceuticals (San Diego, Calif.) at a concentration of 250 mg/mL. Prior to polymerization, HSA was diluted to 25 mg/mL with phosphate buffered saline (PBS) (1.42 g Na₂HPO₄, 8.18 g NaCl, and 0.75 mg KCl per liter, pH=7.4) up to a final volume of 2 L. A 70% glutaraldehyde solution (Sigma Aldrich, Atlanta, Ga.) was then added to HSA solutions at the following molar ratios of glutaraldehyde to HSA: 24:1, 60:1, and 94:1. The polymerization reaction was incubated at 37° C. for 3 hours, then quenched with 25 mL of 1 M sodium borohydride and incubated for 30 minutes. PolyHSA solutions were subjected to diafiltration with a modified lactated Ringer's buffer (115 mM NaCl, 4 mM KCl, 1.4 mM CaCl₂, 13 mM NaOH, 27 mM sodium lactate, and 2 g/L N-acetyl-L-cysteine) on a 100 kDa hollow fiber filter (Spectrum Labs, Rancho Dominguez, Calif.) a total of 4 times and concentrated. The PolyHSA solutions were then filtered through 0.2 μm filters (due to its high MW and viscosity, the 94:1 PolyHSA samples could not be sterile filtered through a 0.2 μm filter) and stored at −80° C. until needed. Polymerizations at each molar ratio of glutaraldehyde:HSA were repeated in triplicate.

SDS-PAGE Analysis—Twenty five micrograms of total protein from each HSA/PolyHSA solution was mixed 1:1 with Lammeli buffer, incubated at 95° C. for 5 minutes, and run on a polyacrylamide gel (12% resolving gel, 4% stacking gel) at 110 V for approximately 1.5 hours. The gel was then stained with Coomassie blue overnight, destained with destaining solution (20% ethanol, 10% acetic acid), and visualized on a Gel Doc XR imaging system (BioRad Hercules, Calif.).

Light Scattering—The absolute MW distribution of HSA/PolyHSA solutions was measured using a SEC column (Ultrahydrogel linear column, 10 μm, 7.8×300 mm, Waters, Milford, Mass.) driven by a 1200 HPLC pump (Agilent, Santa Clara, Calif.), controlled by Eclipse 2 software (Wyatt Technology, Santa Barbara, Calif.) connected in series to a DAWN Heleos (Wyatt Technology) light scattering photometer and an OptiLab Rex (Wyatt Technology) differential refractive index detector. The mobile phase consisted of 20 mM phosphate buffer (pH 8.0), 100 ppm NaN₃, and 0.2 M NaCl (Fisher Scientific) in HPLC grade water that was filtered through a 0.2 μm membrane filter. HSA and PolyHSA solutions were diluted to 1 mg/ml with the mobile phase, and 100 μl of the sample was injected into the column via a 1200 Autosampler (Agilent). All data were collected and analyzed using Astra 5.3 (Wyatt Technology) software.

Circular Dichroism—CD spectra were obtained on an AVIV Circular Dichroism Spectrophotometer Model 202 (Aviv Biomedical Lakewood, N.J.). HSA and PolyHSA solutions were diluted with 20 mM phosphate buffer to approximately 80 μg/mL. The cell temperature was maintained at 25° C. and each spectra was averaged over 3 consecutive measurements taken at 1 nm intervals from 200-250 nm.

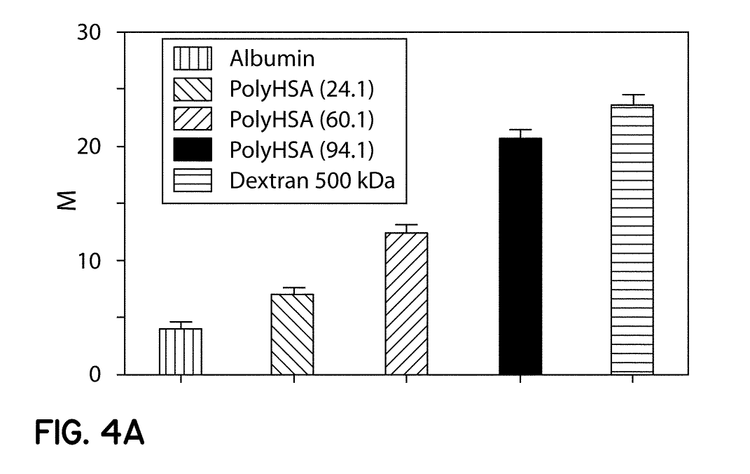

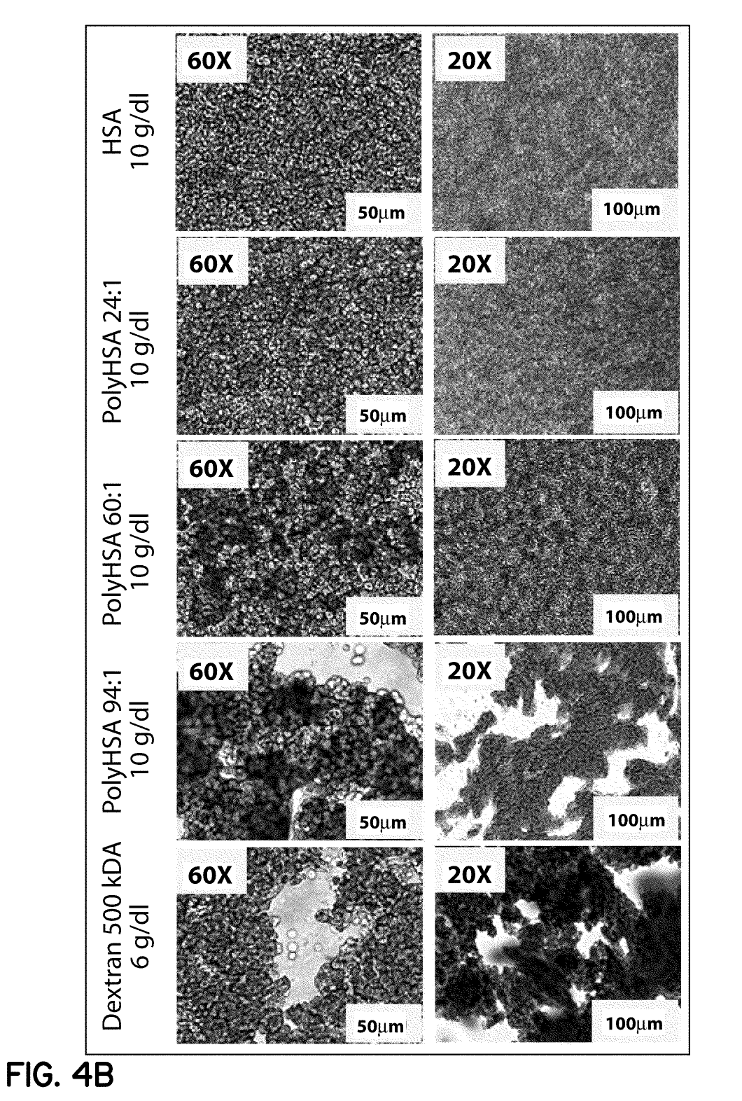

RBC Aggregation—The extent of RBC aggregation in PolyHSA solutions under stasis was measured using a transparent cone-plate shearing instrument that uses the light transmission method (FIG. 4A). The instrument consists of a transparent horizontal plate and rotating cone, between which the blood sample is placed with a light source and photocell arranged vertically (i.e., perpendicular to the plane of the cone and plate) to measure light transmission through the sample. The degree of RBC aggregation was assessed from triplicate measurements on a 0.35 mL sample of heparinized Syrian hamster blood mixed with the test solution at a volume ratio of 1:1, with the photometric rheoscope (Myrenne Aggregometer, Myrenne, Roetgen, Germany). The Myrenne “M” aggregation parameter was determined as follows: The sample was first exposed to a brief period of high shear (600 s⁻¹) to disrupt any preexisting RBC aggregates. The rotation was then stopped, and the light transmittance through the blood sample was recorded for 10 s; the average change in light transmission over this period was taken as the M value (units are arbitrary). If no aggregation occurred, then the light transmission remains constant, and M=0. Aggregation of the RBCs reduces scattering and allows more of the light to reach the photocell, yielding a positive M value, the magnitude of which increases with the degree of aggregation. The use of this technique, as well as comparisons of this index of aggregation (M) with other methods and with different animal species, has been described previously. M indices in 5% HSA (no aggregation) and 6% dextran 500 kDa (aggregation) were measured as control solutions to compare with PolyHSA solutions.

Viscosity and COP Measurements—The viscosity of HSA/PolyHSA solutions was measured in a cone/plate viscometer DV-II plus with a cone spindle CPE-40 (Brookfield Engineering Laboratories, Middleboro, Mass.) at a shear rate of 160/sec, while the COP was measured using a Wescor 4420 Colloid Osmometer (Wescor, Logan, Utah).

Results for the synthesized PolyHSA compositions.

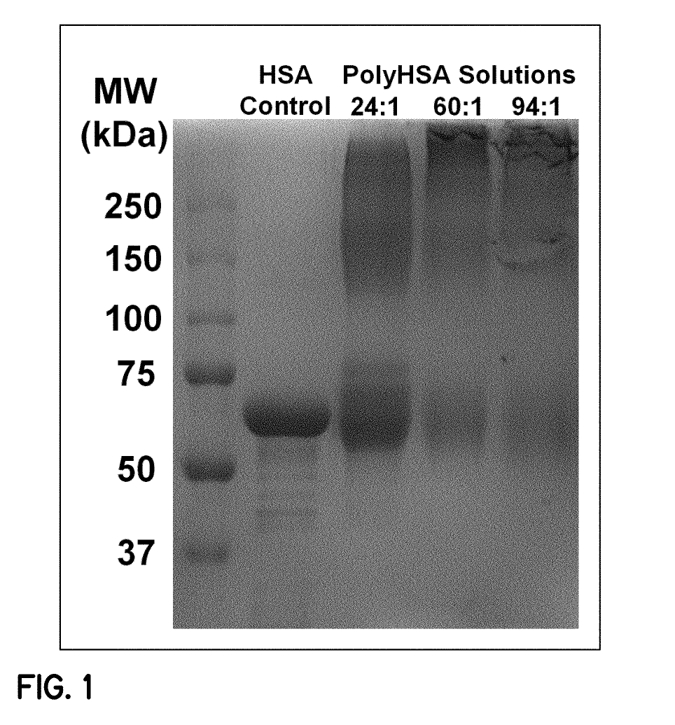

SDS-PAGE Analysis—Polymerization of HSA with glutaraldehyde produced a variety of high MW species, as shown in FIG. 1. Monomeric HSA is included as a control in lane 2, which shows a strong band around 67 kDa (HSA monomer) and several faint bands at lower MWs (degradation products). Each of the PolyHSA solutions show intense broad bands above 100 kDa, corresponding to PolyHSA. Some unpolymerized HSA also remains in each of the PolyHSA solutions, however, the amount of HSA monomer decreases as the glutaraldehyde:HSA molar ratio increases.

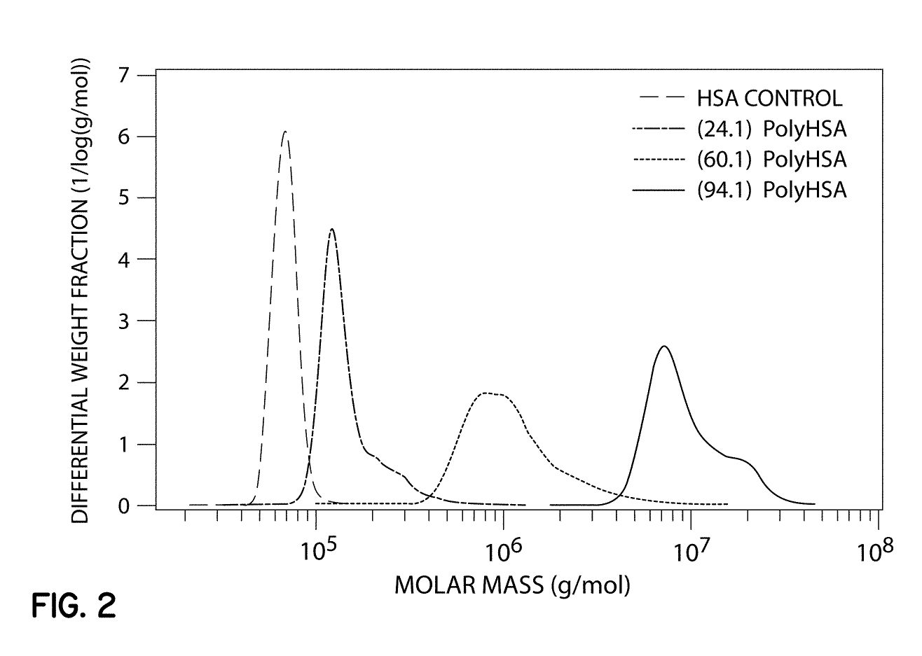

Light Scattering—The MW distributions shown in FIG. 2 reinforce the SDS-PAGE results, showing that the MW of the PolyHSA solutions increases as the molar ratio of glutaraldehyde:HSA increases. The observed weight averaged MW of the HSA control (70 kDa) is very close to the expected MW (67 kDa). Table I shows that polymerization of HSA at every molar ratio of glutaraldehyde:HSA significantly increased the weight averaged MW of the PolyHSA solutions, from a modest change seen with PolyHSA 24:1 (243 kDa) to the large increase observed in PolyHSA 94:1 (11.8 MDa).

TABLE I
Weight Averaged MW of HSA and PolyHSA based
on 3 separate polymerization reactions.

| Solution | Weight Averaged MW (kDa) |
|---|---|
| HSA Control | 70 ± 1 |
| 24:1 PolyHSA | 243 ± 60 |
| 60:1 PolyHSA | 1,997 ± 102 |
| 94:1 PolyHSA | 11,839 ± 2,669 |

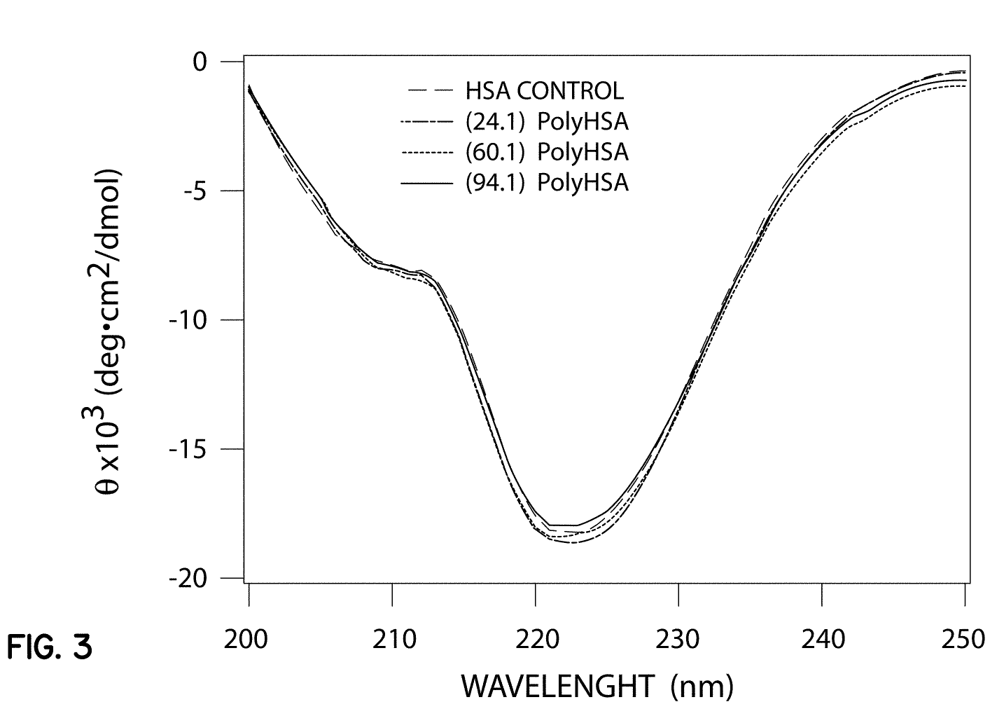

Circular Dichroism—The CD spectra of HSA and each of the PolyHSAs are unaffected by polymerization (FIG. 3). The spectra for monomeric HSA exhibits strong minima around 212 and 224 nm, indicating the presence of alpha helices. These minima indicate the presence of alpha helices, which account for 60% of the secondary structure of HSA The CD spectra of PolyHSA samples are highly similar to HSA, showing strong minima at 212 and 224 nm.

Viscosity and COP—The molar ratio of glutaraldehyde:HSA has a significant effect on the final viscosity and COP of the PolyHSA product, as shown in Table II. In general, the viscosity of PolyHSA solutions seems to increase with increasing glutaraldehyde:HSA molar ratio. The viscosity of each PolyHSA solution is higher than that of monomeric HSA (1.4 cp), ranging from 1.6 cp (PolyHSA 24:1) to 15.2 cp (PolyHSA 94:1), which is a dramatic increase over HSA. In contrast, the COP of the solutions decreases as the molar ratio of glutaraldehyde:HSA increases. A 50% decrease in COP is observed with PolyHSA 24:1, while PolyHSA 60:1 and PolyHSA 94:1 have an almost insignificant COP (4 and 1 mm Hg, respectively).

TABLE II
Viscosity and COP of HSA and PolyHSA solutions
at a concentration of 10 g/dL.

| | Viscosity | COP |
|---|---|---|
| Sample | (cp) | (mm Hg) |
| HSA Control | 1.4 | 42 |
| 24:1 PolyHSA | 1.6 | 22 |
| 60:1 PolyHSA | 11.2 | 4 |
| 94:1 PolyHSA | 15.2 | 1 |

RBC Aggregation—The molar ratio of glutaraldehyde to HSA has a significant effect on RBC aggregation. PolyHSA promotes mild formation of RBC aggregates with different morphologies and sizes depending on the weight averaged MW of the PolyHSA solution (FIG. 4A). RBC aggregate morphology varies from short linear rouleaux at low cross-link densities to continuous RBC networks for PolyHSA 94:1 (FIG. 4B).

Discussion of the results obtained from the synthesis of PolyHSA.

Effects of Polymerization on MW and Secondary Structure—SDS-PAGE analysis and light scattering results show that glutaraldehyde effectively polymerizes HSA at molar ratios of glutaraldehyde:HSA ranging from 24:1 to 94:1 to yield a mixture of high MW PolyHSAs. As expected, increasing the glutaraldehyde:HSA ratio increases the weight averaged MW of the PolyHSA product. The CD spectra also show that the reaction of glutaraldehyde with HSA does not unfold the protein. Therefore, PolyHSA likely retains the beneficial antioxidant and toxin binding properties of monomeric HSA.

Rheological Properties of PolyHSA—The viscosities of all PolyHSA solutions are higher than the viscosity of HSA at the same total protein concentration. This effect is likely due to the large increase in the weight averaged MW of the PolyHSA solutions, which increases the frequency of molecular interactions between neighboring PolyHSA molecules in solution and increases the solution viscosity. In contrast, the COP of the PolyHSA solutions decreases with increasing MW and is significantly lower than that of monomeric HSA. The COP is primarily determined by the presence of unpolymerized HSA and small HSA polymers in solution. Therefore as the cross-link density increases, the concentration of unpolymerized HSA and small HSA polymers decreases. This in turn reduces the COP.

RBC Aggregation by PolyHSA—RBC aggregation experiments show that PolyHSA species with larger MW tend to elicit mild aggregation of RBCs, however, the RBC aggregation effect of the largest PolyHSA studied is still less than that of dextran.

Applications of PolyHSA in Transfusion Medicine—It has been erroneously perceived that lowering blood viscosity leads to an overall health benefit by decreasing peripheral vascular resistance and heart workload. On the contrary, plasma replacement compositions with a high viscosity increase the blood vessel wall shear stress, which induces endothelial cells to produce NO that dilates the blood vessels. Therefore, vascular resistance and heart workload may be decreased in patients with low Hct or blood volume via a high viscosity plasma replacement composition. The viscosity of a plasma replacement composition can be increased by increasing its MW or concentration or a combination of both. However, there are physiological limitations to either approach, since increasing the plasma replacement concentration also increases the COP, while increasing the MW of the plasma replacement composition promotes RBC aggregation. Increasing the COP pulls extravascular fluid into the intravascular space and reduces the blood viscosity to a point lower than the desired value. Likewise, RBC aggregation thickens the marginal zone of the RBC-poor plasma layer and decreases the hydraulic resistance, which decreases the shear rate and lowers the apparent viscosity of blood. In any case, the use of high viscosity plasma replacement compositions must be weighed by the effects of autotransfusion and RBC aggregation. High MW PolyHSA solutions may not be optimal for exchange transfusion of subjects with full Hct, since their high viscosity may increase the vascular resistance. At high (or normal) Hcts, a small increase in plasma viscosity may non-linearly increase the blood viscosity. High MW PolyHSA solutions may be better suited for small volume resuscitation of hemorrhagic shock and in cases of extreme anemia.

This example shows that glutaraldehyde can be used to produce a high MW HSA polymer with conserved secondary structure, high viscosity and low COP. The high MW of the PolyHSA should limit extravasation and its high viscosity should induce vasodilation and increased microvascular perfusion. The low COP may limit volume expansion of PolyHSA beyond the infused volume; however, the exclusion volume of the high MW PolyHSA will insure maintenance of the infused volume.

In cases where volume expansion is desired, there are several possible strategies to remedy the low COP of PolyHSA. PolyHSA can be mixed with a compound having a high COP to create a plasma replacement composition with both high viscosity and high COP to function as a PE. PolyHSA may also be used as an optimal plasma replacement, rather than a PE.

When tested in animal models of extreme hemodilution and hemorrhagic shock, PolyHSA (60:1) described herein resulted in improved systemic and microvascular responses compared to commercial PEs. PolyHSA synthesized at a cross-link density of 60:1 and at a concentration of 10 g/dL, significantly improved cardiac output and microvascular flow during extreme hemodilution compared to dextran 70 kDa (6 g/dL) and HSA (5 g/dL). In addition, PolyHSA (60:1) significantly recovered microvascular flow and blood gases during hemorrhagic shock compared to HSA (10 g/dL) and HES (Hextend™, 6 g/dL).

What is claimed is:

1. A plasma replacement composition comprising a polymerized human serum albumin (PolyHSA) wherein the PolyHSA is stabilized by the reduction of Schiff bases on said PolyHSA.

2. The composition of claim 1 wherein the PolyHSA has a molecular weight (MW) of at least about 100 kDa.

3. The composition of claim 1 wherein the PolyHSA has a cross-linker to human serum albumin (HSA) molar ratio of at least about 10:1.

4. The composition of claim 3 wherein the cross-linker is selected from the group consisting essentially of glutaraldehyde, succindialdehyde, activated forms of polyoxyethylene and dextran, -hydroxy aldehydes, such as glycolaldehyde, N-maleimido-6-aminocaproyl-(2′-nitro,4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl)butyrate, sulfosuccinimidyl 4-(p-maleimidophenyl)butyrate, 1-ethyl-3-(3-dimethylarninopropyl)carbodiimide hydrochloride, N,N′-phenylene dimaleimide, and combinations thereof.

5. The composition of claim 3 wherein the cross-linker is from at least one of a bisimidate class, the acyl diazide class, or the aryl dihalide class.

6. A process for producing a plasma replacement composition comprising:
polymerizing a HSA with a cross-linker, reducing the Schiff bases with a reducing agent, and collecting the PolyHSA.

7. The process of claim 6 wherein the reducing agent is sodium borohydride, sodium cyanoborohydride and other reducing agents.

8. The process of claim 6 wherein the molar ratio of the cross-linker to HSA is at least about 10:1.

9. The process of claim 6 wherein the cross-linker is selected from the group consisting essentially of glutaraldehyde, succindialdehyde, activated forms of polyoxyethylene and dextran, -hydroxy aldehydes, such as glycolaldehyde, N-maleimido-6-aminocaproyl-(2′-nitro,4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl)butyrate, sulfosuccinimidyl 4-(p-maleimidophenyl)butyrate, 1-ethyl-3-(3-dimethylarninopropyl)carbodiimide hydrochloride, N,N′-phenylene dimaleimide, and combinations thereof.

10. The process of claim 6 wherein the cross-linker is from at least one of a bisimidate class, the acyl diazide class, or the aryl dihalide class.

11. The process of claim 6 wherein the collected PolyHSA has a MW of at least 100 kDa.

12. A method of treating a subject with hypovolemia comprising infusing a plasma replacement composition that includes a PolyHSA

13. The process of claim 12 wherein the PolyHSA is free of Schiff bases.

14. The process of claim 12 wherein the PolyHSA has a MW of at least about 100 kDa.

15. The process of claim 12 wherein the PolyHSA has a cross-linker to HSA molar ratio of at least about 10:1.

16. The process of claim 15 wherein the cross-linker is selected from the group consisting essentially of glutaraldehyde, succindialdehyde, activated forms of polyoxyethylene and dextran, -hydroxy aldehydes, such as glycolaldehyde, N-maleimido-6-aminocaproyl-(2′-nitro,4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl)butyrate, sulfosuccinimidyl 4-(p-maleimidophenyl)butyrate, 1-ethyl-3-(3-dimethylarninopropyl)carbodiimide hydrochloride, N,N′-phenylene dimaleimide, and combinations thereof.

17. The process of claim 16 wherein the cross-linker is from at least one of a bisimidate class, the acyl diazide class, or the aryl dihalide class.
